(19) United States
(12) Patent Application Publication
(10) Pub. No.: US 2003/0144472 A1
(43) Pub. Date: Jul. 31, 2003

(54) Method of identifying designable protein backbone configurations

(75) Inventors: Eldon Emberly, Plainsboro, NJ (US); Chao Tang, West Windsor, NJ (US); Ned S. Wingreen, Princeton, NJ (US)

Correspondence Address: SCULLY SCOTT MURPHY & PRESSER, PC, 400 GARDEN CITY PLAZA, GARDEN CITY, NY, 11530

(73) Assignee: NEC Research Institute, Inc., Princeton, NJ

(21) Appl. No.: 10/066,496

(22) Filed: Jan. 31, 2002

(51) Int. Cl. C07K 14/00; C07K 7/08; G06G 7/48; G06G 7/58

(52) U.S. Cl. 530/324; 530/350; 703/11

(57) ABSTRACT

The invention provides a method for identifying new designable protein backbone configurations. The method includes the steps of: (a) specifying a fixed number of secondary structural elements having a set of dihedral angle pairs (b) generating a set of stacks comprising said secondary structural elements; and (c) evaluating designability of said stacks.

### FIELD OF THE INVENTION

The present invention is directed to a method of identifying designable protein backbone configurations.

### BACKGROUND OF THE INVENTION

Proteins are an essential component of all living organisms, constituting the majority of all enzymes and functional elements of every cell. Each protein is an unbranched polymer of individual building blocks called amino acids. In general, there are 20 different natural amino acids, and each protein is a chain of from 50 to 1000 amino acids. Hence there are a vast number of possible protein molecules. A simple bacterium will only employ a few hundred distinct proteins, while it is estimated that there are 50,000 distinct human proteins. In each case, the information for all these proteins is encoded in the DNA of every cell of the organism. By convention, the region of DNA coding for a single protein is called a “gene”. The machinery of the cell interprets the information in the DNA gene to string together the correct sequence of amino acids to form a particular protein. For natural proteins, the amino-acid sequence can be obtained directly from the sequence of DNA bases (A,C,T,G) in the gene for that protein via a known code.

Naturally occurring proteins are composed of two fundamental structural building blocks, alpha-helices and beta-strands. A typical protein structure is a packing of helices and strands connected by turns. The helices and strands are stabilized by the high propensity of some amino acids to form helices and of others to form strands. Because some amino acids are hydrophobic, the helices and strands pack together in a specific way to minimize the exposure of the hydrophobic regions to water. Other interactions, such as hydrogen bonding, can also play a significant role in determining the precise packing arrangement.

In order for a protein to perform its function, the chain must fold into a particular structure. Although there is some apparatus in the cell that assists folding, it is generally accepted that the natural folded structure is the minimum free-energy state of the protein chain. Hence, the information for both the structure and function of each protein is contained in and dependent upon the sequence of amino acids. However, it has proven difficult to predict the folded structure from a knowledge of the amino acid sequence.

Experimentally, the native folded structures of several thousand proteins have been obtained by X-ray crystallographic and/or nuclear magnetic resonance techniques. These methods can often identify the average position in the folded protein of every atom, other than hydrogen, to within 1-2 Angstroms. From this detailed structural information, several general observations about proteins have been made. First, the overall structure of the folded protein is described in terms of the configuration of the backbone plus the orientations of the various amino acid side chains. The backbone configuration is well characterized by the set of dihedral angles, phi and psi, for each amino acid. The covalent bond lengths and three-atom bond angles are found to vary little among structures. Second, within the natural backbone configurations there is a preponderance of specific folds or “secondary structures”. These are alpha helices and beta strands, with loops connecting these fundamental building blocks together. A plot of the frequency of occurrence of particular dihedral angle pairs is called a Ramachandran plot. The prevalence of beta strands and alpha helices is clearly indicated by the high frequency of phi-psi pairs in the angular regions associated with these two folds. Finally, the secondary structures may be packed together in many different ways. The arrangement of these secondary structural elements, with the connecting loops cut away, is generally known as the protein's “stack”. The stack, plus information about which elements are connected to other elements by loops, is known as the tertiary structure of the protein. Therefore, the tertiary structures of two proteins are considered to be the same if both contain the same sequence of secondary structures packed together in the same overall spatial orientation. In accordance with the present invention, “tertiary structure” and “fold” are synonymous and may be used interchangeably.

Among the known natural structures, several hundred qualitatively distinct tertiary structures or folds have been identified. Indeed, it has been estimated that there are roughly 2000 distinct protein folds in nature. Despite the variety of protein sizes, shapes, and backbone configurations represented in the known folding topologies, it remains an open problem to design novel protein folds.

An important consideration in the design of novel protein folds is thermodynamic stability. Stability puts minimum requirements on the size of folds. In nature, proteins of more than approximately 50 amino acids can be stabilized by the formation of a core of hydrophobic amino acids. Chains of fewer than 50 amino acids generally require additional stabilizing factors such as covalent disulfide bonds, strong salt bridges, or metal cofactors such as the zinc ion in zinc fingers. A method for designing new protein structures with more than 50 amino acids is therefore more likely to produce stable folds than a method restricted to shorter chains.

One motivation behind the design of new protein folds is that such design would offer a new strategy for the creation of pharmaceutical drugs, including antibiotics. Other biological roles for proteins with new folds include acting as pesticides and herbicides. Proteins act as catalysts of inorganic as well as organic reactions, and may have industrial applications in this role. Proteins are also known to play a role in inorganic synthesis as in bones, teeth, and shells, and applications of new protein folds in inorganic chemistry and material engineering can be envisioned. The ability to design new folds could also prove instrumental in developing methods to predict the folding of natural proteins, the so-called “protein folding problem”.

Two major accomplishments of intelligent protein design are the synthesis of a zinc finger without zinc (Dahiyat et al. (1997) Science, 278(5335):82-7) and that of a right-handed coiled coil (Harbury et al. (1998) Current Opinion in Struc. Bio., 9(4):509-513). Both of these achievements of design represent small modifications of naturally occurring structures.

In designing the modified zinc finger FSD-1, Dahiyat et al. began with the known backbone configuration of the naturally occurring zinc-finger protein Zif268. They applied an algorithm that tested many possible amino-acid sequences, and many possible side-chain orientations, to find a sequence with particularly low energy when its backbone adopted the exact backbone configuration of Zif268. It was confirmed by nuclear magnetic resonance that the redesigned zinc finger FSD-1 folded into the predicted structure. The important property of FSD-1 compared to the natural protein Zif268, is that FSD-1 no longer depended on a zinc ion for stability.

The structures designed and synthesized by Harbury et al. are all coiled coils, i.e. dimers, trimers, or tetramers of alpha helices superhelically twisted about each other. Harbury et al. were able to design sequences of amino acids so that the superhelical twist of these coiled coils was right handed, in contrast to the left handed twist most commonly found in nature. (A naturally occurring right-handed coiled-coil dimer is known (MacKenzie et al. (1997) Science, 276(5309):131-3.) The methods employed are very specific to the coiled-coil class of structures. Specifically, only a single family of parametrically related backbone configurations was considered. There is no evident way to generalize the Harbury et al. approach to classes of structures other than the coiled coil.

A method for protein design has been described by Miller and coworkers (U.S. patent application Ser. No. 09/730,214, incorporated herein by reference) in which backbones are generated as a sequence of particular pairs of dihedral angles. All backbone configurations which can be made from a chosen set of dihedral angle-pairs are generated. In order to generate a sufficient variety of configurations, the number of pairs of dihedral angles must be at least 3. The number of configurations generated is therefore at minimum 3\hat{ }N, where N is the number of amino acids in the chain. This exponential growth of the number of configurations with the length of the chain limits the method to chains of fewer than thirty amino acids, given current computational limits.

Knowledge exists to optimize a sequence for a predetermined backbone configuration. However, there is no existing method of identifying new designable protein backbone configurations of more than thirty amino acids. The approach of Dahiyat et al. can only reproduce naturally found configurations. The approach of Harbury et al. can only produce close variants of a particular natural configuration, the coiled coil. The approach of Miller et al. is limited to chains of fewer than thirty amino acids.

Moreover, experimental approaches to designing new protein structures have severe limitations. Studies of the folding of random amino-acid sequences by Davidson and Sauer, (Proc. Natl. Acad. Sci., USA, (1994) 91(6):2146-50)identified some sequences which appear to fold. However, the conformations were not sufficiently rigid to allow structural determination by either X-ray crystallography or nuclear magnetic resonance techniques. Without even an approximate knowledge of the folded structure, no systematic progress could be made to increase rigidity.

Recently, Szostak and colleagues ((2001) Nature 410:715-718) have been able to find folding proteins by in vitro evolution. This method, however, can only be used to identify proteins which bind to a particular substrate. It is also a random process, and there is no guarantee that the proteins found in this way have novel folds.

Thus, backbone configurations employed to date have either been taken directly from nature, or are slight modifications of natural configurations, or are limited to chains of fewer than thirty amino acids. The ability to identify foldable backbone configurations of new protein folds. Thus, there exists a need in the art to identify new designable protein structures, particularly for chains of more than thirty amino acids.

### SUMMARY OF THE INVENTION

Therefore it is an object of the present invention to provide a method for identifying designable protein backbone configurations having more than thirty amino acids. The methods of the present invention provide a technique for the systematic enumeration of all of the possible stacks of secondary protein structures, such as alpha helices and beta strands. The elements of the stack are chosen depending on the size and type of protein desired. The stacks are clustered and the designability of the stacks is determined.

The method of the present invention for identifying designable protein backbone configurations having more than thirty amino acids comprises the steps of (a)specifying a fixed number of secondary structural elements having a set of dihedral angle pairs; (b) generating a set of stacks comprising the secondary structural elements; and (c) evaluating designability of each stack within a set of stacks.

Preferably, the method further comprises the step of assessing the completeness of the stack. The method preferably also further comprises the step of grouping the stacks into clusters.

### BRIEF DESCRIPTION OF THE DRAWINGS

FIGS. 1(a) and 1(b) are representative fits for two SCOP (Structural Classification of Proteins) proteins to model four-helix bundles generated by the method of the invention.

FIG. 2 is a histogram of the number of structures with a given designability for the representative structures of the four-helix-bundle ensemble. Only a few of the structures are highly designable. Most structures are lowest energy states of few or no sequences.

FIG. 3 Illustrates four the most designable four-helix folds. FIG. 3(a) is an up and down fold. FIG. 3(b) is an up and down with a cross-over connection fold. FIG. 3(c) is an X repressor-type fold. FIG. 3(d) is an orthogonal array fold.

FIG. 4(a) is a surface area exposure for each of the four helices for structure (a) in FIG. 3. FIG. 4(b) is a calculated hydrophobic-polar patterning of each of the four helices.

FIG. 5 is a best fit of surface distribution of the 11 SCOP proteins to top 100 designable structures found using h₀=+2K_{B}T.

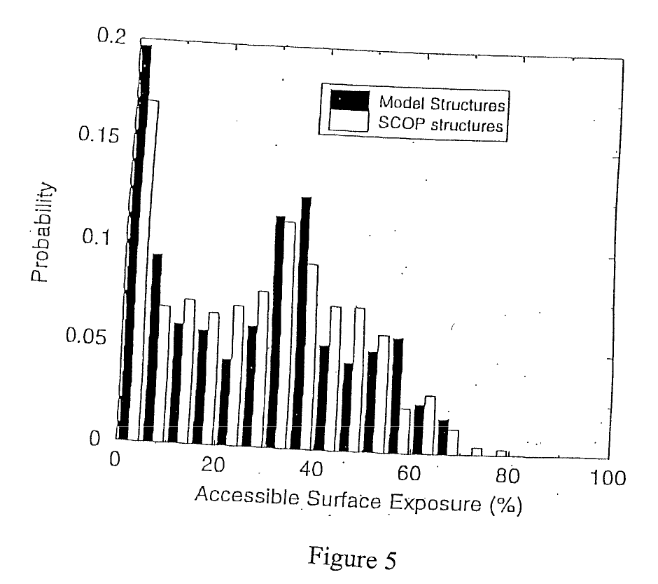

### DETAILED DESCRIPTION OF THE INVENTION

The invention provides a method-for identifying new designable protein folds. The present invention contemplates a method for the generation of stacks of secondary structures. The stacks of secondary structures will, in accordance with the present invention, facilitate the design of protein folds which are not seen in nature.

The present invention contemplates the design of sequences of real amino acids which will adopt a target configuration. The stacks which are targeted for the design of new protein structures are those stacks belonging to clusters with the largest cluster designabilities. Sequences designed to fold into these configurations are expected to exhibit protein-like folding properties. The vast number of possible configurations makes generation of a complete set impractical for chains of more than thirty amino acids. However, by considering stacks of secondary structural elements, it is possible to restrict the number of configurations that must be considered. This is because sections of a protein chain can be forced to adopt alpha-helical or beta-strand folds by choosing only amino acids with high alpha-helical or beta-strand propensities for these sections. It is then only necessary to consider the possible packings of a fixed set of secondary structural elements. The method described by the present invention therefore contemplates designing stacks using a fixed set of secondary structural elements. The resulting computational simplification, for the first time, permits the design of novel folded structures consisting of many more than thirty amino acids.

The pattern of surface exposure along a protein chain is believed to dominate the folding of proteins found in nature. That is, a particular sequence will generally adopt the fold that leaves the hydrophobic (“water fearing”) amino acids of the sequence buried in the core of the fold. Therefore, in accordance with the present invention, the pattern of surface exposure of each configuration or stack, once determined, provides a useful measure of protein folding properties. In the method disclosed herein, a “configuration” refers to a particular spatial arrangement of secondary structural elements, such as alpha-helices and beta-strands, having a specified order in which the elements are to be connected by loops. By “stack” is meant the packing of secondary structural elements, with the connecting turns cut away. The stack, plus information about which elements are connected together by turns, yields the protein's fold.

By “designability” is meant the number of amino acid sequences that can fold into a particular stack. A “highly designable fold” is a fold that is the ground state of an unusually large number of amino-acid sequences, i.e. the number of amino acid sequences that have a particular stack as their lowest energy conformation. In accordance with the present invention, the amino acid sequences associated with designable folds are expected to have protein-like folding properties, i.e. thermodynamic stability, stability under changes of amino acids, and fast folding. Designable folds are identified by first specifying a fixed number of alpha-helices and/or beta-strands of fixed lengths. For example, in accordance with the present invention, one to twenty alpha-helices and/or beta strands can be specified. In accordance with the Example provided herein, four alpha-helices, each helix having fifteen amino acids, are specified.

Once a fixed number of secondary structures are specified, the present invention contemplates the systematic enumeration of all of the possible stacks of such structures. The elements of the stack are selected depending on the size and type of protein desired. For example, a four-helix protein, having fifteen amino acids per alpha-helix is selected. Each element in the stack is assumed to be a rigid body, described by its center of mass and three Euler angles. The element itself is specified by its alpha-carbon-atom positions and its amino acid side-chain centroids, the latter taken to lie in the direction of the beta-carbon atom at a distance of 2.1 Angstroms from the alpha-carbon atom. These alpha-carbon atom positions and amino acid side-chain centroids are determined by the backbone dihedral angles, which are about {φ,Ψ,}={−60, −50} for an alpha-helix.

An initial stack is generated by first randomly selecting the center of mass and Euler angles for each element. However, if any alpha-carbon-atom or centroid of one element passes too close to an alpha-carbon or centroid of another element in space (i.e. self-avoidance), then that configuration will be energetically unfavorable for any possible sequence of amino acids. Therefore, if an element's center of mass and Euler angles cause it to violate self-avoidance with one of the other elements, then its degrees of freedom are randomly re-selected. Then these variables are relaxed so as to minimize the packing energy.

A local minimum of the packing energy is found using a conjugate gradient method described in Numerical Recipes (Press et al. Chapter 10, Numerical Recipes in C. Cambridge University Press 1992, incorporated herein by reference). The choice of packing energy is motivated by the hydrophobic force, which produces the compact stacks found in nature. The first term of the packing energy is

E₁=Σᵢsᵢ

where sᵢ is the surface exposure of the ith amino acid along the chain. The surface exposure of each amino acid is calculated by approximating each side chain as a sphere with radius R_{S}=3.1 Angstroms centered at a distance L=2.1 Angstroms from its alpha-carbon atom, in the direction of the beta-carbon. The surface exposure sᵢ of each side-chain sphere is found using the method of Flower (supra), with a water molecule represented as a sphere of radius R_{H20}=1.4 Angstroms.

A second term is then added which represents the effect of excluded volume. This term E₂ is a pairwise repulsive energy between backbone alpha-carbon atoms and centroids on different elements. This excluded volume energy is given by,

$${E}_{2} = - {V}_{0} {\sum }_{ij} [ {( 2 {r}_{CA} / {r}_{Ai , j} )}^{12} + {( 2 {r}_{CB} / {r}_{Bi , j} )}^{12} + {( ( {r}_{CA} + {r}_{CB} ) / {r}_{ABi , j} )}^{12} ]$$

where r_{CA} and r_{CB} are sphere sizes for the backbone alpha-carbon atoms and centroids respectively, r_{Ai,j} is the distance between backbone alpha-carbon atoms i and j, r_{Bi,j} is the distance between centroids i and j, and r_{ABi,j} is the distance between backbone alpha-carbon atom i and centroid j. V₀ sets the scale of the repulsive energy. In one embodiment r_{CA}=1.75 Angstroms and r_{CB}=2.25 Angstroms.

Finally, a weak compression energy E₃ and an energy E₄ due to tethers between the ends of connected elements are included. These energies have the form,

E₃=0.5Kr_{g}²,

where r_{g} is the radius of gyration of the entire stack, and

E₄=Σᵢ0.5K_{S}(d_{i,j}−d0_{i,j})²,

where, d_{i,j} is the distance between the connected ends of tethered elements i and j, and d0_{i,j} is a specified equilibrium length. The spring constants, K and K_{S} are chosen to be small so that these terms act as weak perturbations.

The actual minimization of the total energy E_{packing}=E₁+E₂+E₃+E₄ using the conjugate gradient method proceeds in steps, akin to annealing. The scheduled parameter is V₀. Initially V₀ is chosen to be large, so that there is a large repulsion between all the elements. (The starting value of V₀ varies depending on the number and size of the chosen elements. The initial V₀ is chosen so as to generate a smooth collapse of the elements.) In accordance with the present invention an initial V₀ is contemplated to be 10-500. At a given V₀, a minimum of E_{packing} is found for the full set of center of mass and angle variables. V₀ is then reduced by a constant factor (i.e. about 90%) and a small random change is made to each degree of freedom. The size of the random change is also scaled along with V₀, with the initial change being 1 Angstrom for the centers of mass and 15 degrees for each Euler angle. The V₀ schedule is terminated when any two centroids are at a distance less than some specified contact distance, usually taken to be 2*R_{S}. At this point, E₃ is set to zero. V₀ is then set to its final value and the last conjugate gradient minimization is performed to yield final values of each rigid element's center of mass and orientation angles. (The final value of V₀ is determined by fitting to a naturally occurring backbone that is composed of similar elements. The fitting procedure is to minimize the coordinate root mean square (crms) between the natural backbone of the elements and the backbone of the same elements after a conjugate gradient minimization using different values of V₀.) This yields a stack.

With the centers of mass and angles determined, various symmetry operations are then performed to generate a plurality of additional stacks wherein each stack is based on a distinct set of randomly selected starting coordinates, such as Euler angles and centers of mass, for example. For alpha-helical elements these are screw operations which correspond to rotating the helix by 100 degrees and translating it by +/−1.5 Angstroms along the helix direction. For beta strands, slide operations are performed which correspond to translating each residue up or down by one residue along the strand direction. Each stack is then checked to see if it satisfies user-supplied constraints. A user-supplied constraint is also understood in accordance with the present invention to mean a predetermined criterion for reducing the number of stacks in a set. For instance, stacks that exceed a specified total surface exposure or have end-to-end distances of connected elements which exceed some cut-off, are excluded from the set. For example, if an end-to-end distance of connected elements within a stack exceeds 12 Angstroms then that stack is excluded from the set.

If a stack satisfies the user-supplied constraints, the surface exposure of each amino acid to water is determined using the method of Flower et al. (Journal of Molecular Graphics and Modelling, 1997 15(4):238-44, incorporated herein by reference) and the structure of the stack and the list of exposures are recorded. Stacks are generated in this way until the ensemble of possible stacks for this model is formed. Each set of stacks is then assessed for completeness.

Designability is determined via a competition for amino-acid sequences within a “complete” set of stacks. Since the method for generating stacks is based on random sampling, a criterion must be specified for determining where to stop sampling. A set of stacks is considered to be “complete” where a specified fraction (about 95%) of newly generated stacks lies within a specified coordinate root mean square (crms) (e.g. about 1.5 Angstroms) of at least one stack already in the set. The distance measure, crms, is defined as

$${crms}^{2} = 1 / N {\sum }_{i} {[ {r}_{i}^{( s )} - {r}_{i}^{( {s}_{1} )} ]}^{2}$$

where rᵢ^{(s)/(s}^{₁}⁾ is the position of the ith alpha-carbon for the (s)/(s₁) stack and N is the number of backbone alpha-carbons. The stacks s and s¹ are aligned by performing a least-squares fit using crms as the metric.

Once the stacks are complete, the stacks are clustered and evaluated for designability. Designable folds are built around the most designable stacks by connecting the elements in the stacks with loops consisting of hydrophilic amino acids of high flexibility (e.g. glycine). In accordance with the present invention, it is possible to ensure that the secondary structural elements in the stacks will form as expected by choosing amino acids which possess a high alpha-helical or beta-strand propensity for these elements.

In the determination of the designability of configurations, those configurations with similar patterns of surface exposure are considered to compete. However, two configurations which are very similar in their total geometry should not be considered as competing folds, but rather as variants of the same fold. Hence, if two stack configurations are sufficiently similar in their three-dimensional arrangement, then they are considered to be members of a single cluster. The following method is a preferred way of grouping stacks into clusters.

In accordance with the present invention it is computationally advantageous to reduce the sample by retaining only one member (i.e. stack) of each cluster. These representative stacks are selected in the following way. The entire set of stacks is sorted according to total surface exposure, i.e. from the most compact to least compact. Starting at the top of this list with the most compact stack, all stacks that are closer to it than 1.5 Angstroms crms are eliminated. This process is repeated for the next most compact structure in the list until the end of the list is reached. A large ensemble of stacks can be compressed by a factor of about 3, to 5.

In accordance with the present invention, all stack configurations within a cluster are treated as variants of a single stack configuration. The designabilities of all configurations within each cluster are summed, and the total is considered to be the designability of the cluster.

In accordance with the present invention, the designabilities of the representative stacks in the complete set, after clustering, are determined by allowing the representative stacks to compete for a random sample of possible amino acid sequences. The “designability” of a stack is defined as the number of amino acid sequences for which that stack has the lowest energy.

To determine the energies of different amino acid sequences on the stacks in the complete set, each amino acid sequence is reduced to the series of hydrophobicities of its individual amino acids. Hydrophobicity is a term representing the free-energy cost of bringing a particular substance in contact with water. It is assumed therefore that the hydrophobic energy is the dominant term contributing to the energy on a given structure.

A preferred expression for the energy of a sequence folded into a particular configuration is

E_{designability}=−Σᵢjᵢs, (1)

where hᵢ is the hydrophobicity of the ith element of the sequence and sᵢ is the surface exposure of the ith amino-acid sphere in the particular stack. For each sequence considered, the stack with the lowest energy given by Eq. (1), is recorded i.e. the ground-state configuration for that sequence is recorded. It is not necessary to find the ground-state configuration for all sequences. By sampling a large number of randomly selected sequences, it is possible to reliably estimate the designabilities of different stacks.

For the designability calculation, binary sequences consisting of only two types of amino acids are employed. Such sequences are known as “HP-sequences”, for hydrophobic (H) and polar (P) amino acids. In accordance with the present invention, a random sequence of amino acids can have a length of 2ⁿ, where n=1-500. The two hydrophobicity values are hᵢ=h₀+δ h, where h₀ is a compactification energy, and δ h measures the relative distance between hydrophobic and polar residues. Using the Miyazawa-Jernigan matrix (S. Miyazawa and R. L. Jernigan (1985) Macromolecules 18:534; S. Miyazawa and R. L. Jernigan (1996) J. Mol Biol 256:623, incorporated herein by reference), incorporated herein by reference, of amino acid interaction energies, a typical energy difference between hydrophobic and polar residues is inferred to equal 1.5 k_{B}T/contact. On average, a buried residue makes four non-covalent contacts. Therefore 26h=6.0 k_{B}T. The compactification energy, ho, is determined by fitting the surface-area distribution of a set of natural m-element bundles to the surface-area distributions for the 50-1000 most designable m-element-stacks, wherein m=1-20, using different values of ho to assess designability. In one embodiment, ho, is determined by fitting the surface-area distribution of a set of natural four-helix bundles to the surface-area distributions for the 100 most designable four-helix-stacks, using different values of h₀ to assess designability. The best fit preferably corresponds to h₀=2 k_{B}T and hydrophobic residues have a hydrophobicity of 5 k_{B}T and polar residues −1k_{B}T.

In another embodiment, the method of the present invention can be generalized to allow flexibility of the secondary structural elements, the alpha-helices and beta-strands. In natural protein structures, alpha helices are relatively rigid, while beta strands are more flexible. Hence, the extension of the method to include flexible elements is more important in the case of beta strands.

The internal flexural modes of rod shaped objects are bending, stretching, and twisting. All these internal flexural modes can be included in the method for both alpha helices and beta strands. It is possible to determine the appropriate degree of flexibility for each internal mode by reference to known protein structures. A preferred method is to extract multiple examples of alpha helices and beta strands from the Protein Structure Database, reduce their alpha-carbon coordinates to vectors, and perform a principal component analysis of the resulting set of vectors (separately for alpha helices and beta strands). This analysis reveals the primary flexural modes, with appropriate weights. A harmonic energy function E_{flex} for these flexural modes can then be added to the packing energy, with coefficients chosen to reproduce the degree of flexibility observed in natural proteins. For example, if the degree of bending of an alpha helix is represented by the angle theta, then the additional term in E_{packing} representing this mode would be

Eₜₕₑₜₐ=cₜₕₑₜₐ(theta)²,

where the constant cₜₕₑₜₐ can be chosen so that the average degree of bending <theta²> in the generated stacks matches that observed in natural structures.

In natural proteins, beta strands are typically stabilized by the formation of hydrogen bonds between strands. To generate stack configurations which include beta strands it is therefore preferable to include an inter-strand hydrogen bonding energy E_{HB} in the packing energy E_{packing}. The skilled artisan can readily evaluate hydrogen-bonding energies between the atoms of a protein backbone, including the case of hydrogen bonding between two beta strands (Gordon et al. (1999) Current Opinion in Struc. Bio., 9(4):509-513).

Thus, where flexible alpha helices and/or beta strands are employed in generating stacks, the energy E_{flex} associated with the flexural modes can be included in E_{designability}. This adds a sequence-independent energy to each stack configuration.

Where beta strands are employed in generating the stacks, the energy E_{HB} associated with hydrogen bonds between beta strands can be included in E_{designability}. This adds a sequence-independent energy to each stack configuration.

The highly designable stack configurations identified in this way are excellent targets for novel protein fold design. First, there will be many possible sequences which will fold into these configurations because of the mutational stability of highly designable configurations. Second, the associated sequences will have few traps, which implies both thermodynamic stability of the ground state and fast folding kinetics. A “trap” is a low energy configuration other than the true ground state. The scarcity of traps follows because it is only configurations with similar patterns of surface exposure that are potential traps for a well-designed sequence. By construction, designable configurations are found in low density regions of configuration space, which means there are few configurations with similar surface-exposure patterns. Thus, all the folding properties normally attributed to real proteins such as mutational stability, thermodynamic stability, and fast folding, can be associated with those sequences having highly designable ground-state configurations.

Methods of designing a sequence of amino acids for a known backbone configuration are known (Dahiyat et al. (1997). Science, 278(5335):82-7, incorporated herein by reference). The method of the present invention does not explicitly generate the backbone configuration for the loops connecting the stack elements, but this has already been achieved and is well within the ken of the ordinary skilled artisan (See, e.g. Vita et al. (1999) PNAS, 96(23) 13091-13096; Liang et al. (2000) Biopolymers, 54:515-523; and Nakajima et al. (2000) Mol. Biol. 296:197-216, each of which are incorporated herein by reference).

In accordance with the present invention predetermined sequences of real amino acids are synthesized according to established methods (see e.g. Dahiyat et al. (1997).

Ultimately, the folded structure of amino acid sequences is determined in accordance with known methods such as using X-ray crystallography and/or nuclear magnetic resonance techniques.

The protein backbone configurations identified in accordance with the present invention offer great promise for the discovery of new pharmaceutical drugs. Proteins are generally noncarcinogenic and nonmutagenic, and nontoxic in their breakdown products. New structures imply qualitatively new functions and have the potential for unanticipated medical benefits.

The newly identified protein structures may also be a source of new antibiotics, pesticides, herbicides, fungicides, etc. Furthermore, the proteins designed in accordance with the present invention can be used as catalysts for inorganic reactions. In nature, proteins are also employed in the fabrication of complexly ordered inorganic structures such as bones, teeth, and shells. Recently, proteins have also been employed in nonbiological fabrication, such as templating of the inorganic synthesis of gold crystallites (Brown et al. (2000) Journal of Molecular Biology, 299(3):725-35. Therefore, the new structures provided by the invention will allow novel applications of proteins in inorganic catalysis and synthesis. Furthermore, production of the protein structures identified by the method of the present invention can take advantage of existing expertise in generating high yields of specific proteins, using either chemical or biological production strategies.

### EXAMPLE

A stack generation method was applied to the packing of four alpha-helices. Each helix was chosen to be fifteen residues long, with backbone dihedral angles {φ,Ψ}={−60°, −50°}. The backbones of turns connecting the helices were not specified, but the turns were constrained to be short. Specifically, a stack was discarded if any of the end-to-end distances between connected helices exceeded 12 Angstroms. The method generated a “complete” ensemble of four-helix stacks consisting of 1,297,808 stacks. This large ensemble of stacks was then clustered, resulting in 188,538 representative stacks.

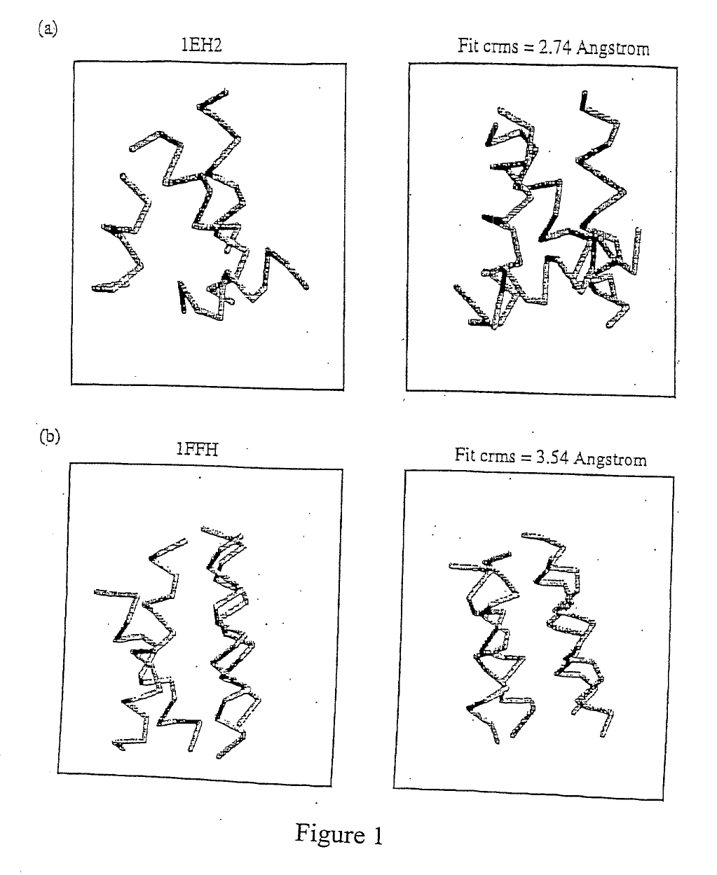

To test if the method reproduced the natural four-helix bundles, 11 proteins with short turns were selected from different Structural Classification of Proteins (SCOP) families, and the representative stacks were searched for the best fits. To account for length differences between helices in the SCOP structures (the lengths ranged from 7-18 residues) and the fifteen-residue helices in the experiment, the shorter length for each comparison was chosen. For the longer helix of each mismatched pair, all possible truncations down to the shorter length were tried. Thus, for each pairing of a SCOP structure with one of the representive stacks, the best fit was computed among all possible combinations of truncations. FIG. 1 shows two overall best fits among all possible pairings. For the 11 natural four-helix bundles, the average crms to a representative stacks was 2.86 Angstroms. A 0.5-1.0 Angstrom background error in each fit due to deviations from {φ,Ψ}={−60°, −50°} in the natural helices was estimated. This estimate was accomplished by computing the crms between a helix constructed using {φ,Ψ}={−60°, −50°} and each helix from the selected SCOP structures. Table I summarizes the results of fitting the natural four-helix bundles to our representive stacks. In all cases, the natural structure had a counterpart in the representative ensemble at a crms distance of less than 3.6 Angstroms per residue.

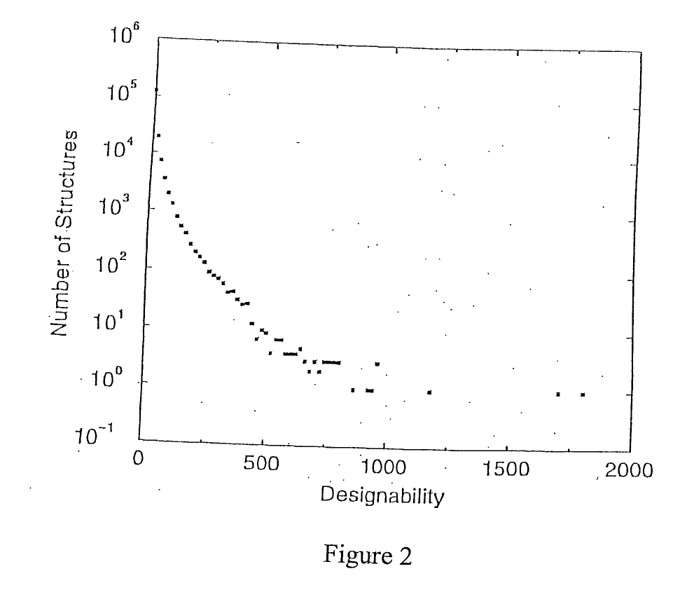

An important goal was to identify stacks with no natural counterparts as candidates for the design of novel protein folds. To identify which stacks might be promising candidates, a designability calculation was performed using a hydrophobic energy on the ensemble of representatives of the four-helix structures. A random sample of 4,000,000 binary amino-acid sequences was used. FIG. 2 shows the results of the designability calculation. The distribution of designabilities is consistent with previous results for both lattice and off-lattice models namely, there is a small set of highly designable structures with the great majority of structures poorly designable or undesignable. The average designability, that is, the average number of sequences per stack, was 4,000,000/188,538=21. The most designable structure was the lowest energy state of 1813 sequences.

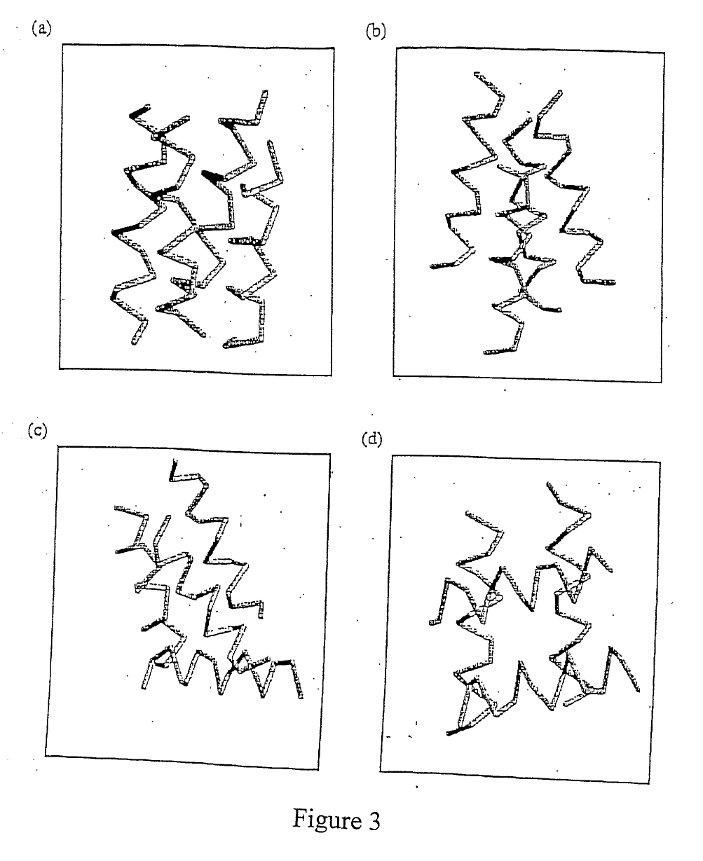

All of the designable stacks fall within one of four folds, and these are shown in order of designability rank in FIG. 3. A metric based on helical directions was used to determine that all of the representative structures with a designability greater than 100 fall within approximately 15°/helix of one of these four folds). The topmost designable structure is an up-and-down four-helix bundle. The second most designable fold is a variant of the up-and-down fold except that there is a crossover connection. The third most designable fold falls within the λ repressor-like DNA-binding domain class. The last fold is an orthogonal array. Table II presents binary sequences which have these structures as lowest energy folds. These particular sequences were calculated by matching them to the surface area pattern of each of the four folds and then performing a simple energy gap optimization. The energy of optimization was done by first calculating the mean surface area exposure of each side chain for each structure. For a given structure, the sequence was then assigned by putting hydrophobic residues on sites which had surface exposures below the mean and polar residues on those sites whose exposure exceeded the mean. The energy gap optimization was then performed. The energy gap was defined to be the energy difference between the ground state energy and the first excited state that at a crms greater than 4 Angstroms (i.e. a structure that is significantly different than the ground state). Point mutations were randomly performed on the sequence by changing an H to a P or a P to an H, and the mutation was maintained if it made the gap larger.

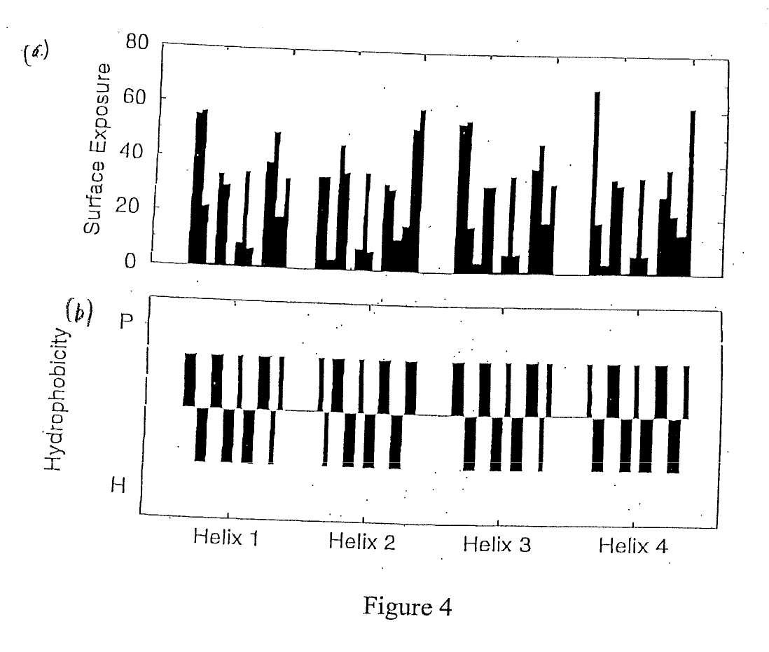

This process of mutations was performed until a sequence was obtained where a mutation in any site made the gap lower. The result of this method is depicted for structure (A) of FIG. 3 in FIG. 4. FIG. 4A shows the pattern of surface exposure along each helix. FIG. 4B is the corresponding calculated hydrophobic-polar patterning of the surface area pattern. The last column in Table II is the energy gap between the ground state structure and the first different fold in the energy spectrum (the low lying excited states all fall within the same fold type).

TABLE I
Results of fitting selected set of 11 proteins from SCOP
database to ensemble of model four helix bundles.

| PDB ID | crms (Angstroms) |
|---|---|
| 1FLX | 2.96 |
| 1FFH | 3.54 |
| 1E6I | 2.85 |
| 1CB1 | 1.65 |
| 1CEI | 2.95 |
| 1A24 | 2.85 |
| 1POU | 2.81 |
| 1AU7 | 3.02 |
| 1EH2 | 2.74 |
| 1IMQ | 2.75 |
| 1DNY | 3.44 |

TABLE II
Results for the top four distinct designable folds for the
model four helix bundles shown in FIG. 3. Column 2 gives the
hydrophobic-polar patterning of each of the length 15 helices.
The last column gives the energy gap in kT between the
structures and their nearest distinct structural
competitor.

| Structure | Sequence | Energy Gap (kT) |
|---|---|---|
| a helix 1 | PPHHPPHHPHHPPHP | |
| a helix 2 | PHPPHHPHHPPHHPP | |
| a helix 3 | PPHMPPEHPHHPPHP | 3.80 |
| a helix 4 | PHHPPHHPHHPPHHP | |
| b helix 1 | PHPPHHPHHPPHHPP | |
| b helix 2 | PHHPPHHPHHHPHHP | |
| b helix 3 | PPHPPHHPHHPPHHP | 2.60 |
| b helix 4 | PPHHPPEHPHHPPHP | |
| c helix 1 | HPHHPPHHPHHPPHP | |
| c helix 2 | PHPPHHPPHHPPHHP | |
| c helix 3 | PHHPHHHPHHHPPPP | 2.65 |
| c helix 4 | PPPPHHPHHPPHHPP | |
| d helix 1 | HPPHHPHHPPHHPPP | |
| d helix 2 | PHHPPHHPHHPPPHP | |
| d helix 3 | PHHPHHPPHHPHHPP | 2.95 |
| d helix 4 | PPHHPHHPPHHPPHP | |

While the invention has been particularly shown and described with respect to illustrative and preferred embodiments thereof, it will be understood by those skilled in the art that the foregoing and other changes in form and details may be made therein without departing from the spirit and scope of the invention that should be limited only by the scope of the appended claims.

In the claims:

1. A method for identifying designable protein backbone configurations comprising:
a. specifying a fixed number of amino acid secondary structural elements;
b. generating a set of stacks comprising said secondary structural elements; and
c. evaluating designability of each stack within said set of stacks.

2. The method of claim 1, wherein said secondary structural elements comprise at least one alpha helix, at least one beta strand or both.

3. The method of claim 1, wherein one secondary structural element corresponds to an alpha helix.

4. The method of claim 1, wherein one secondary structural element corresponds to beta strand.

5. The method of claim 1, wherein said fixed number of secondary structural elements is one to twenty.

6. The method of claim 5, wherein said fixed number of secondary structural elements is four.

7. The method of claim 2, wherein said alpha helix is about 15 amino acids in length.

8. The method of claim 1, wherein a center of mass and an Euler angle are randomly selected for each element of said stack.

9. The method of claim 1, wherein step (b) includes generating an initial stack by a conjugate gradient method.

10. The method of claim 9, wherein the conjugate gradient method includes a step of determining the minimum packing energy of said stack.

11. The method of claim 9, further including a step of generating additional stacks by performing one or more symmetry operations.

12. The method of claim 11, wherein said symmetry operations comprise slide operations or screw operations.

13. The method of claim 1, wherein step (b) further includes a step of confirming that said stack does not exceed a predetermined constraint wherein a stack that exceeds said predetermined constraint is discarded.

14. The method of claim 13, wherein said predetermined constraint is an end-to-end distance between connected helices.

15. The method of claim 1, wherein step (b) further includes a step of determining the surface exposure of each amino acid within each stack to water.

16. The method of claim 9, wherein a plurality of stacks are generated, wherein each stack is based on a distinct set of randomly selected starting coordinates.

17. The method of claim 16, wherein said randomly selected starting coordinates include a center of mass and Euler angles for each element of said stack.

18. The method of claim 9, wherein further including a step of assessing the completeness of said plurality of generated stacks.

19. The method of claim 18, wherein plurality of generated stacks is complete when about 90% to about 95% of newly generated stacks lie within a root-mean-square distance of about 1.5 Angstroms of at least one stack already in the set.

20. The method of claim 1, further comprising a step of grouping said set of stacks generated in step (b) into clusters.

21. The method of claim 19, wherein said clustered stacks are sorted and listed according to total surface exposure to water from the most compact stack to the least compact stack.

22. The method of claim 21, where in all stacks that are within 1.5 Angstroms crms of said most compact stack are eliminated from said cluster and wherein said process is repeated for a next most compact stack on said list until the end of said list is reached.

23. The method of claim 1, wherein a random set of amino acid sequences is generated based on binary sequences consisting of Hydrophobic (H) and Polar (P) amino acids wherein a random sequence of amino acids has a length of 2ⁿ wherein n=1-500.

24. The method of claim 23, wherein each amino acid sequence is reduced to the hydrophobicities of its individual amino acids.

25. The method of claim 21, wherein each amino acid in each stack has a surface exposure value.

26. The method of claim 23, wherein the energy of an amino acid sequence folded into a particular configuration is
E_{designability}=Σᵢhᵢsᵢ,
where hᵢ is the hydrophobicity of the ith element of the sequence and sᵢ is the surface exposure of the ith amino-acid sphere in the particular stack.

27. The method of claim 26, wherein for each random amino acid sequence considered, the stack with the lowest energy is a designable structure.

28. The method of claim 26, wherein a highly designable stack is identified when the number of amino acid sequences with said stack as the lowest energy state, is larger than the average number of sequences per stack.
